(19) United States
(12) Patent Application Publication
(10) Pub. No.: US 2025/0032811 A1
(43) Pub. Date: Jan. 30, 2025

(54) IMPLANTABLE CARDIOVERTER DEFIBRILLATOR DEVICE COMPRISING AN ANTENNA ARRANGED ON A LEAD

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 18/716,181

(22) PCT Filed: Dec. 6, 2022
(86) PCT No.: PCT/EP2022/084520

§ 371 Date: Jun. 4, 2024

(30) Foreign Application Priority Data
Dec. 14, 2021 (EP) 21214307.7

(51) Int. Cl. A61N 1/39; H01Q 1/22

(52) U.S. Cl. CPC A61N 1/3956; H01Q 1/2291

(57) ABSTRACT

An implantable cardioverter defibrillator device comprises a generator device comprising a shock generation circuitry for producing an electrical shock pulse for performing a defibrillation therapy and a communication circuitry for establishing a communication connection to an external device in a frequency range above 2 GHz. At least one lead comprises a shock electrode for emitting said electrical shock pulse. The at least one lead comprises at least one antenna operatively connected to the communication circuitry for transmitting communication signals to and/or receiving communication signals from the external device in said frequency range above 2 GHz.

### CROSS-REFERENCE TO RELATED APPLICATIONS

This application is the United States National Phase under 35 U.S.C. § 371 of PCT International Patent Application No. PCT/EP2022/084520, filed on Dec. 6, 2022, which claims the benefit of European Patent Application No. 21214307.7, filed on Dec. 14, 2021, the disclosures of which are hereby incorporated by reference herein in their entireties.

### TECHNICAL FIELD

The instant invention concerns an implantable cardioverter defibrillator device and a method for operating an implantable cardioverter defibrillator device.

### BACKGROUND

An implantable cardioverter defibrillator device generally comprises a generator device comprising a shock generation circuitry for producing an electrical shock pulse for performing a defibrillation therapy and a communication circuitry for establishing a communication connection to an external device. The implantable cardioverter defibrillator device furthermore comprises at least one lead comprising a shock electrode for emitting said electrical shock pulse.

The implantable cardioverter defibrillator device in particular is designed for emitting electrical shocks in case life-threatening arrhythmias of a patient's heart are detected. By means of an electrical shock a defibrillation shall be achieved in order to reset the cardiac rhythm back to a normal state.

For example, within a home monitoring system, the implantable cardioverter defibrillator device shall communicate with an external device in order to receive, e.g., configuration data from the external device or to transmit processing data, for example indicative of an operation of the implantable cardioverter defibrillator device, to the external device. The external device may serve as a relay for receiving data from and/or transmitting data to a home monitoring service center, at which data may be processed and analyzed, e.g., for assessment by a physician.

Conventionally, dedicated communication techniques are used for establishing a communication between an implantable medical device and an external device, which rests outside of the patient. For such communication techniques, for example a MICS protocol may be employed, using a transmission of (electromagnetic) signals at a frequency around 400 MHz.

At the frequency of the MICS band a communication connection may be established to implanted devices which rest at a substantial implantation depth. There however is a desire to use other communication techniques which are widely available, such as Bluetooth or Bluetooth Low Energy (BLE), allowing for an increased data throughput, but utilizing a frequency range well above the MICS band. By using a standardized communication technique widely used in the field of consumer electronics, it may become possible to establish a connection to an implanted device using a common communication device, such as a smart phone or a tablet computer, hence easing the setup of a home monitoring system. At increased frequencies, however, a signal damping due to body tissue increases, such that a communication connection potentially cannot be easily and reliably established to an implanted device at a substantial implantation depth.

In an implantable cardioverter defibrillator device there may be limitations for the placement of the generator device, making it potentially impossible to adapt an implantation depth in order to allow for a communication using, e.g., Bluetooth or Bluetooth Low Energy. For example, the generator device may typically be implanted left laterally between the musculus serratus anterior and the musculus latissimus dorsi, posing restrictions on a required implantation depth.

U.S. Publication No. 2020/0197710 A1 discloses methods, systems and devices for wireless signal transmission employing a fractal antenna coupled with a lead. For example, the fractal antenna may be placed, in the context of a deep brain stimulation operation, between a patient's skull and its scalp and may convey signals to the lead.

The present disclosure is directed toward overcoming one or more of the above-mentioned problems, though not necessarily limited to embodiments that do.

### SUMMARY

It is an object of the instant invention to provide an implantable cardioverter defibrillator device and a method for operating an implantable cardioverter defibrillator device which allow in an easy and efficient way to establish a communication to an external device.

In one aspect, an implantable cardioverter defibrillator device comprises a generator device comprising a shock generation circuitry for producing an electrical shock pulse for performing a defibrillation therapy and a communication circuitry for establishing a communication connection to an external device in a frequency range above 2 GHz. The implantable cardioverter defibrillator device further comprises at least one lead comprising a shock electrode for emitting said electrical shock pulse. The at least one lead comprises at least one antenna operatively connected to the communication circuitry for transmitting communication signals to and/or receiving communication signals from the external device in said frequency range above 2 GHz.

The implantable cardioverter defibrillator device comprises a generator device having a communication circuitry, for example a communication chip, which is configured to establish a communication connection to an external device in a frequency range above 2 GHz. Herein, for transmitting signals or for receiving signals within the context of the communication connection, the communication circuitry is connected to one or multiple antennas enabling signal transmission and/or reception.

As restrictions may exist for implanting the implantable cardioverter defibrillator device, in particular such that the generator device may, in an implanted state, have to be placed at a substantial implantation depth, the antenna is not placed on the generator device, for example on a housing of the generator device, but is arranged on a lead which, in an operational state, is connected to the generator device and extends from the generator device. The lead carries a shock electrode for performing a regular therapeutic function of the implantable cardioverter defibrillator device, namely, to emit a shock pulse for performing a defibrillation therapy.

A lead for performing a therapeutic function hence also is used for establishing a communication connection to an external device. Whereas the generator device, for example, subcutaneously is implanted in a patient, for example at an implantation location between the musculus serratus anterior and the musculus latissimus dorsi, the lead extends from the generator device towards a location of interest such that the shock electrode arranged on the lead is placed within the patient at an implantation site to perform a desired therapeutic function, in particular a defibrillation function.

At increased frequencies, signal damping by body tissue is increased. By placing the antenna on the lead the antenna is spatially removed from the generator device, such that the placement of the antenna in an implanted state of the implantable cardioverter defibrillator device is not restricted by the implantation of the generator device. The antenna hence may be placed at an implantation depth which is sufficiently small in order to establish a reliable communication connection in a frequency range above 2 GHz.

The implantable cardioverter defibrillator device may in particular be a non-transvenous implantable cardioverter defibrillator device (in short non-transvenous ICD), which is designed for implantation external to a patient's heart. In a non-transvenous implantable cardioverter defibrillator device, a generator device may, for example, be implanted subcutaneously in a patient. A lead, in a connected state, extends from the generator device, the lead being implanted such that it fully rests outside of the patient's heart. The lead may, for example, extend from the generator device towards a location in the region of the patient's sternum, the shock electrode hence being placed outside of the patient's heart for emitting an electrical shock pulse at a location external to the patient's heart.

The term “non-transvenous” in this respect in particular shall express thatthe lead of the non-transvenous implantable cardioverter defibrillator device does not extend transvenously into the heart, but fully rests outside of the patient's heart.

In another embodiment, the implantable cardioverter defibrillator device may be designed for a transvenous implantation, i.e., by implanting a lead to extend transvenously into the heart.

The implantable cardioverter defibrillator device generally is configured to emit a shock pulse for achieving a defibrillation. The implantable cardioverter defibrillator device may serve for monitoring and treating potentially life-threatening arrhythmias of a patient's heart.

If the implantable cardioverter defibrillator device is a non-transvenous implantable cardioverter defibrillator device, the shock electrode in an implanted state of the defibrillator device is placed outside of the heart of the patient, for example in the region of the sternum of the patient, such that a shock pulse for achieving a defibrillation is generated outside of the heart.

In one embodiment, the at least one antenna is placed on an intermediate portion of the at least one lead in between a proximal end of the at least one lead facing the generator device and the shock electrode. At the proximal end the lead, in an implanted state, is connected to the generator device. The lead extends from the generator device and may be flexibly bendable such that it may be guided from the generator device towards an implantation site at which the shock electrode is placed for performing a therapeutic function. The at least one antenna serving to establish a communication connection to an external device herein is placed on an intermediate portion in between the proximal end and the shock electrode. The at least one antenna hence is placed on such a portion of the lead which rests, in an implanted state, within the body in between the generator device and the shock electrode.

The intermediate portion, at implantation according to a prescribed use, may be configured for implantation at an implantation depth smaller than an implantation depth of the generator device. During implantation the generator device may be implanted such that it is received in a pocket left laterally in between the musculus serratus anterior and the musculus latissimus dorsi. The lead extends from the generator device such that the intermediate portion comes to lie at a location which beneficially is close to (but beneath) the patient's skin. The antenna on the lead hence may lie at a shallow location depth, such that signals in a frequency range above 2 GHz may penetrate through body tissue to and from the antenna for establishing a communication connection without experiencing an excessive signal damping.

The antenna is, for example, enclosed within a lead body of the lead. The lead body may, for example, be made from a silicone material and may be flexibly bendable. Also the antenna may herein be flexibly deformable, for example when the antenna is formed by an electric wire.

In one embodiment, the at least one antenna is connected to a shielded connection line extending along the at least one lead from the at least one antenna towards the generator device. The antenna may, for example, be formed by a wire. The connection line serves to connect the antenna to the communication circuitry of the generator device, wherein the connection line is shielded in order to allow for a signal transmission between the antenna and the communication circuitry at low signal loss.

Generally, the antenna may be designed for a dedicated signal transmission and reception in a frequency range above 2 GHz. In one embodiment, the antenna may be formed by a wire portion. In another embodiment, the antenna may, for example, be formed by a patch antenna. The antenna may have a certain directivity. Alternatively, the antenna may be omnidirectional.

In one embodiment, the at least one antenna is formed by a wire portion having a length in between 2 cm and 10 cm, preferably between 3 cm and 5 cm. The length of the antenna may be adapted to a desired wavelength. For example, a frequency band employed for a Bluetooth or Bluetooth Low Energy communication is the 2.4 GHz ISM spectrum band (2400 MHZ to 2483.5 MHz). The length of the antenna may be adapted to, e.g., a quarter wavelength at a desired frequency, wherein propagation characteristics (in particular a dielectric constant) in body tissue may be taken into account.

In one embodiment, the communication circuitry is configured to establish a communication connection to the external device in a frequency range between 2 GHz and 3 GHZ and/or between 5 GHz and 6 GHz. In particular, the communication circuitry may be configured to establish a communication connection to the external device in a frequency range between 2.4 GHz and 2.5 GHZ, specifically in the 2.4 GHz ISM spectrum band (2400 MHz to 2483.5 MHz).

In one embodiment, the communication circuitry is configured to establish a communication connection to the external device using Bluetooth or Bluetooth Low Energy. For example, the Bluetooth 5.2 standard may be employed.

As another communication technology, for example WiFi may be employed, using a frequency band at 2.4 GHz and/or 5 GHz.

In one embodiment, the generator device comprises a first connector and the at least one lead comprises a second connector connectable to the first connector for connecting the at least one lead to the generator device. The connectors comprise contact elements for establishing an electrical connection in between the generator device and the lead. By means of the electrical connection in particular sense signals as received by electrode poles arranged on the lead may be transmitted to a processing circuitry of the generator device, and shock energy for emitting a shock pulse may be fed to the shock electrode arranged on the lead.

In addition, in one embodiment, the first connector and the second connector may be configured to establish an operative connection between the at least one antenna arranged on the at least one lead and the communication circuitry of the generator device. Hence, communication signals are transmitted via the connection established between the lead and the generator device by means of the connectors. The connectors may, e.g., be standardized IS4/DF4 connectors allowing for a releasable connection of the lead to the generator device.

In one embodiment, the generator device comprises a further, second antenna operatively connected to the communication circuitry for transmitting communication signals to and/or receiving communication signals from the external device in said frequency range above 2 GHz. Whereas a first antenna is placed on the lead, a second antenna may be placed on the generator device. The second antenna likewise allows for a signal transmission and/or reception in the frequency range above 2 GHz.

The second antenna may allow for establishing a communication connection between the generator device and the external device, for example, in a state in which the implantable cardioverter defibrillator device is not yet implanted in a patient, for example when the lead is not yet connected to the generator device. Alternatively or in addition, the multiple antennas may allow for a multiple input multiple output (MIMO) signal reception and transmission to improve quality and data rate of the communication.

In one embodiment the implantable cardioverter defibrillator device comprises a sensing arrangement for sensing electrocardiogram signals. Sensed signals are forwarded to a processing circuitry of the generator device, which processes the signals in order to, e.g., identify a ventricular contraction event in a sensed electrocardiogram signal. Based on a sensed cardiac activity, a therapeutic function may be performed.

The sensing arrangement may comprise multiple electrode poles. One or multiple electrode poles of the sensing arrangement herein may be placed on the lead carrying the shock electrode. For example, one electrode pole may be placed on the lead at a position proximal to the shock electrode. Another electrode pole may be placed on the lead at a position distal to the shock electrode.

Further electrode poles may be placed on further leads connected to the generator device. Alternatively or in addition, one or multiple electrode poles may be formed by a housing of the generator device. Yet alternatively or in addition, the shock electrode may be used as a sense electrode pole for sensing electrocardiogram signals.

In one embodiment, the sensing arrangement comprises three or more electrode poles. The three or more electrode poles form multiple pairs of electrode poles which may be used for sensing electrocardiogram signals. The different pairs span sense vectors which, each by itself, may be used to sense an electrocardiogram signal. The different sense vectors herein may exhibit a different spatial sensitivity with respect to electrocardiogram signals and hence may be used to sense information in a multichannel processing. Signals received by means of the different sense vectors as spanned by different pairs of electrode poles may be combined in order to sense ventricular activity and to derive information from electrocardiogram signals.

In one embodiment, the processing circuitry is configured to identify ventricular contraction events in an electrocardiogram signal to determine a timing of a therapeutic action. Ventricular contraction events may be sensed in electrocardiogram signals of one or of multiple signal vectors as spanned by one or multiple pairs of electrode poles. Information from multiple signals received by multiple pairs of electrode poles spanning different sense vectors may in particular be combined in order to reliably detect ventricular contraction events in electrocardiogram signals.

In another aspect, in a method for operating an implantable cardioverter defibrillator device the implantable cardioverter defibrillator device comprises a generator device having a shock generation circuitry for producing an electrical shock pulse for performing a defibrillation therapy. The method comprises: establishing, using a communication circuitry of the generator device, a communication connection to an external device in a frequency range above 2 GHz, wherein communication signals are transmitted to and/or received from the external device in said frequency range above 2 GHz using at least one antenna operatively connected to the communication circuitry and arranged on at least one lead connected to the generator device, the at least one lead comprising a shock electrode for emitting said electrical shock pulse.

The advantages and advantageous embodiments described above for the implantable cardioverter defibrillator device equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the present invention shall subsequently be described in more detail with reference to the embodiments as shown in the drawings. Herein:

FIG. 1 shows a schematic drawing of an implantable cardioverter defibrillator device in an implanted state in a patient;

FIG. 2 shows the cardioverter defibrillator device of FIG. 1, illustrating sense vectors spanned by different pairs of electrode poles of a sensing arrangement of the device:

FIG. 3 shows a schematic drawing of an implantable cardioverter defibrillator device in an implanted state in a patient comprising a generator device and a lead, an antenna for signal reception and/or transmission in a frequency range above 2 GHz being arranged on the lead; and

FIG. 4 shows a schematic drawing of connectors of the generator device and the lead.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

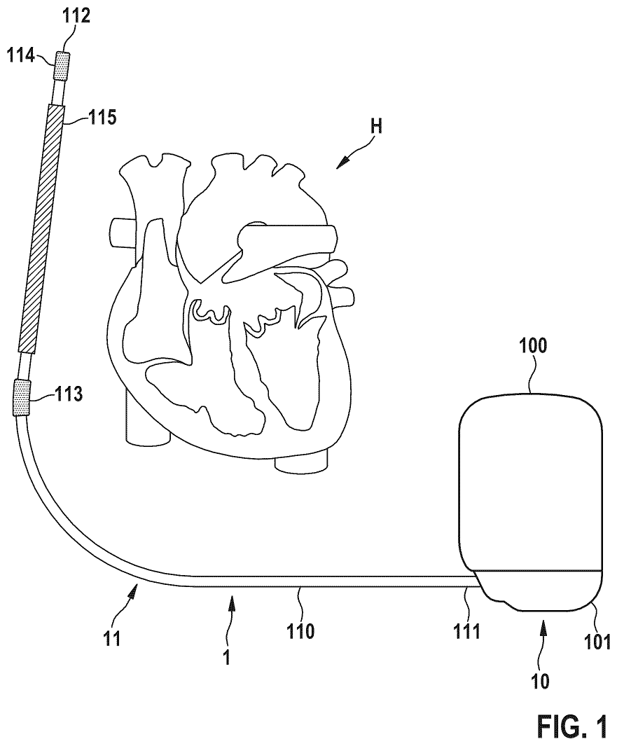

Referring to FIG. 1, in a setup of a therapy system an implantable cardioverter defibrillator device 1 is designed as a non-transvenous implantable cardioverter defibrillator device 1. The non-transvenous implantable cardioverter defibrillator device 1 is implanted such that the implantable cardioverter defibrillator device 1 is completely external to the heart H, the implantable cardioverter defibrillator device 1 comprising a generator device 10 encapsulated within a housing 100, and a lead 11 connected to the generator device 10 at a proximal end 111 and carrying electrode poles 113, 114 as well as a shock electrode 115 in the shape of a coil formed on a distal portion close to a distal end 112 of the lead 11. The electrode poles 113, 114, for example formed as ring electrodes on either side of the shock electrode 115, serve to sense cardiac signals for processing within the generator device 10 of the implantable cardioverter defibrillator device 1, such that based on sensed signals an arrhythmia may be identified and a shock pulse may be generated for providing for a defibrillation therapy.

The implantable cardioverter defibrillator device 1, in the embodiment of FIG. 1, is designed for a non-transvenous implantation, that is an implantation external to the patient's heart H.

In particular, the lead 11 connected to the generator device 10 shall rest outside of the patient's heart H and shall not extend transvenously into the heart, the shock electrode 115 hence, in an implanted state, being placed outside of the heart H for providing for a defibrillation therapy.

For example, the generator device 10 may be implanted subcutaneously in a patient. The lead 11, with a lead body 110, may extend from the generator device 10 towards the sternum of the patient, the lead 11, for example, tunneling through tissue in the region of the sternum and being placed beneath the sternum of the patient.

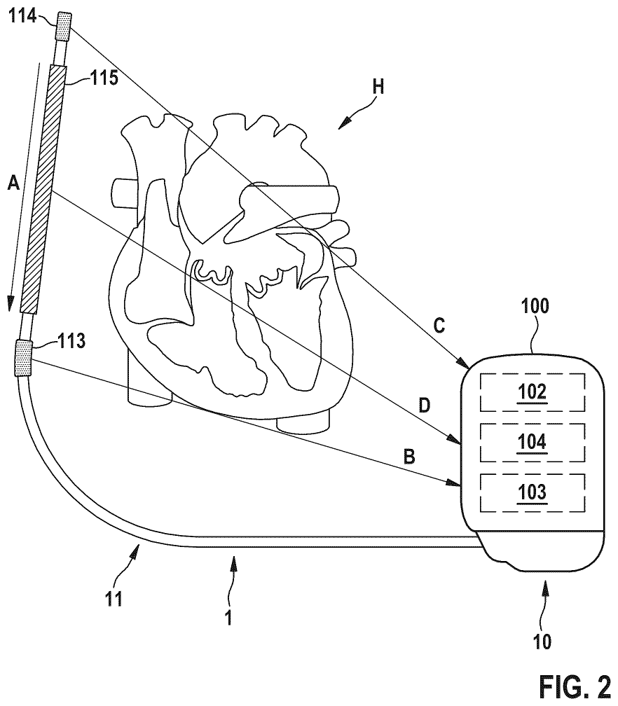

Referring now to FIG. 2, the generator device 10 generally comprises a processing circuitry 102 for controlling operation of the implantable cardioverter defibrillator device 1. In addition, the generator device 10 comprises a shock generation circuitry 103 and an energy storage 104, in particular in the shape of a battery.

The processing circuitry 102 in particular serves to process signals sensed via a sensing arrangement formed by the electrode poles 113, 114 arranged on the lead 11 and additional poles, such as the shock electrode 115 and the housing 100 of the generator device 10. The different poles of the sensing arrangement form pairs of electrode poles in between which sense vectors A, B, C, D are spanned, as illustrated in FIG. 2, the different sense vectors A, B, C, D allowing to sense electrocardiogram signals from the patient's heart H with a different spatial sensitivity, hence allowing to sense and process information from the patient's heart H in a multichannel processing.

The implantable cardioverter defibrillator device 1, as shown in FIGS. 1 and 2 in particular, shall be configured to perform a defibrillation therapy.

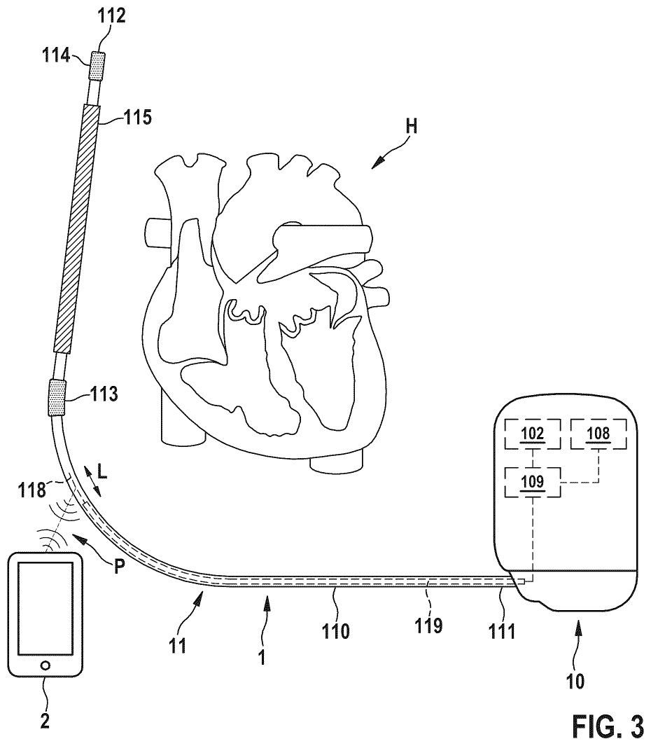

Referring now to FIG. 3, the implantable cardioverter defibrillator device 1 comprises a communication circuitry 109 for establishing a communication connection P with an external device 2, for example within a home-monitoring system.

The external device 2 may, for example, be a consumer communication device, such as a smart phone or a tablet computer. Both the communication circuitry 109 and a communication circuitry of the external device 2 herein may be configured to set up a communication connection P according to a standardized communication protocol, for example employing Bluetooth, Bluetooth Low Energy or WiFi.

Within the implantable cardioverter defibrillator device 1 as shown in FIG. 3, a communication with the external device 2 shall take place in a frequency range above 2 GHz, for example in the 2.4 GHz ISM band used by Bluetooth or Bluetooth Low Energy. In that a communication takes place by using a standardized communication technology as employed by and as implemented on consumer communication devices, a communication connection P between the implantable cardioverter defibrillator device 1 and the external device 2 may be easily established using a widely available communication technique, hence reducing the requirements for a dedicated adaptation of the external device 2 for communicating with the implantable cardioverter defibrillator device 1.

For establishing a communication, the communication circuitry 109 of the generator device 10, for example a communication chip for Bluetooth or Bluetooth Low Energy communication, is connected to an antenna 118 arranged on the lead 11. The antenna 118, for example, is formed by a wire portion having a defined length L adapted to a specific frequency range, for example the 2.4 GHz ISM band for communication using Bluetooth or Bluetooth Low Energy. The antenna 118 in particular may have a length L in between 2 cm and 10 cm, for example between 3 cm and 5 cm.

The antenna 118 on the lead 11 is connected to the communication circuitry 109 of the generator device 10 by a shielded connection line 119, which extends from the antenna 118 along the lead 11 towards the proximal end 111 of the lead 11 and at the proximal end 111 is electrically connected to the generator device 10.

By arranging the antenna 118 on the lead 11, the placement of the antenna 118 becomes independent of the implantation of the generator device 10. In particular, the generator device 10 of the implantable cardioverter defibrillator device 1 may be implanted by placing it in a pocket left laterally in between the musculus serratus anterior and the musculus latissimus dorsi, posing restrictions on a required implantation depth of the generator device 10. The shock electrode 115, which is arranged in the region of the distal end 112 of the lead 11, in turn is implanted at an implantation site, for example in the region of the sternum of the patient, for example beneath the sternum. An intermediate portion of the lead 11 in between the proximal end 111 and the shock electrode 115 may however be implanted such that the antenna 118 rests at a small implantation depth, for example close to (but beneath) the skin of the patient.

By placing the antenna 118 on an intermediate portion of the lead 11 in between the proximal end 111 and the shock electrode 115, signal transmission and reception via the antenna 118 may take place at shallow tissue penetration and hence at reduced signal damping, such that a reliable communication connection P in between the implantable cardioverter defibrillator device 1 and the external device 2 may be established using the antenna 118 in an implanted state of the cardioverter defibrillator device 1.

The generator device 10, in one embodiment, comprises an additional, second antenna 108 connected to the communication circuitry 109. The second antenna 108 may allow for establishing a communication connection P to an external device 2, for example, in a state in which the cardioverter defibrillator device 1 is not yet implanted. Alternatively or in addition, by using the multiple antennas 108, 118 a multiple input multiple output (MIMO) communication may be established to improve communication quality and data throughput.

The lead 11 is formed flexible such that it may flexibly be arranged and implanted within the patient. The lead 11 in particular is flexibly bendable. A lead body 110, for example, may be formed from a silicone material. The antenna 118 beneficially is enclosed within the material of the lead body 110.

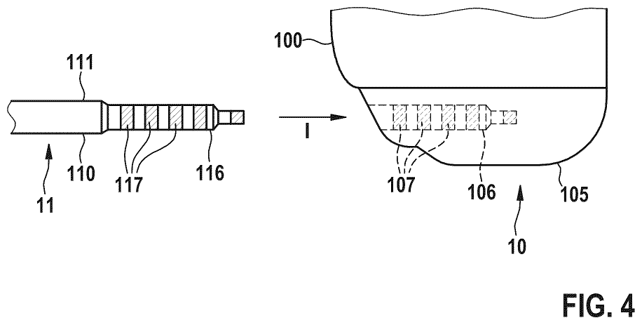

Referring now to FIG. 4, in one embodiment the lead 11, at its proximal end 111, comprises a connector 116 having a multiplicity of contact elements 117, for example according to the definition of an IS4/DF4 connector. The connector 116 of the lead 11 may be plugged into, along an insertion direction I, a corresponding connector 106 in the shape of a plug receptacle formed on a connection block 105 of the generator device 10. The connector 106 comprises contact elements 107 for electrically contacting with the contact elements 117 of the connector 116 such that, by plugging the connectors 106, 116 into each other, a mechanical as well as an electrical connection in between the lead 11 and the generator device 10 is established.

In one embodiment, the connectors 106, 116 also are configured to establish an electrical connection between the antenna 118 arranged on the lead 11 and the communication circuitry 109. By means of the contact elements 107, 117 hence also the antenna 118 arranged on the lead 11 is electrically connected to the communication circuitry 109.

The idea underlying the present invention is not limited to the embodiments described above, but may be implemented in an entirely different fashion.

An implantable cardioverter defibrillator device may comprise one or multiple leads, with one or multiple electrode poles arranged on each lead.

The implantable cardioverter defibrillator device may use Bluetooth, Bluetooth Low Energy or another wireless communication technology, in particular as regularly employed by consumer communication devices such as smart phones or tablet computers, for establishing a communication to an external device. Communication herein takes place in a frequency range above 2 GHz, wherein the communication circuitry may specifically be adapted for communication using a communication technology in a particular frequency band, such as a frequency band at 2.4 GHz or 5 GHz.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Non-transvenous implantable cardioverter defibrillator device
10 Generator device
100 Housing
101 Connection block
102 Processing circuitry
103 Shock generation circuitry
104 Energy storage (battery)
105 Connector block
106 Connector
107 Contact elements
108 Antenna device
109 Communication circuitry
11 Electrode lead
110 Lead body
111 Proximal end
112 Distal end
113, 114 Electrode pole
115 Shock electrode (coil)
116 Connector
117 Contact elements
118 Antenna
119 Shielded connection line
2 External device
A-D Reception vector
H Heart
I Insertion direction
L Length
P Communication link

1. An implantable cardioverter defibrillator device, comprising:
a generator device comprising a shock generation circuitry for producing an electrical shock pulse for performing a defibrillation therapy and a communication circuitry for establishing a communication connection to an external device in a frequency range above 2 GHz; and
at least one lead comprising a shock electrode for emitting said electrical shock pulse;
wherein the at least one lead comprises at least one antenna operatively connected to the communication circuitry for transmitting communication signals to and/or receiving communication signals from the external device in said frequency range above 2 GHz.

2. The implantable cardioverter defibrillator device according to claim 1, wherein the implantable cardioverter defibrillator device is a non-transvenous implantable cardioverter defibrillator device.

3. The implantable cardioverter defibrillator device according to claim 1, wherein the at least one antenna is placed on an intermediate portion of said at least one lead in between a proximal end of the at least one lead facing said generator device and the shock electrode.

4. The implantable cardioverter defibrillator device according to claim 3, wherein the intermediate portion is configured, at implantation according to a prescribed use, to be implanted at an implantation depth smaller than an implantation depth of the generator device.

5. The implantable cardioverter defibrillator device according to claim 1, wherein the at least one antenna is connected to a shielded connection line extending along the at least one lead from the at least one antenna towards the generator device.

6. The implantable cardioverter defibrillator device according to claim 1, wherein the at least one antenna is formed by a wire portion having a length in between 2 cm and 10 cm, preferably between 3 cm and 5 cm.

7. The implantable cardioverter defibrillator device according to clailm 1, wherein the communication circuitry is configured to establish a communication connection to the external device in a frequency range between 2 GHz and 3 GHZ and/or between 5 GHz and 6 GHz.

8. The implantable cardioverter defibrillator device according to claim 1, wherein the communication circuitry is configured to establish a communication connection to the external device in a frequency range between 2.4 GHz and 2.5 GHZ.

9. The implantable cardioverter defibrillator device according to claim 1, wherein in the communication circuitry is configured to establish a communication connection to the external device using Bluetooth or Bluetooth Low Energy.

10. The implantable cardioverter defibrillator device according to claim 1, wherein the generator device comprises a first connector and the at least one lead comprises a second connector connectable to the first connector for connecting the at least one lead to the generator device.

11. The implantable cardioverter defibrillator device according to claim 10, wherein the first connector and the second connector are configured to establish an operative connection between the at least one antenna arranged on the at least one lead and the communication circuitry of the generator device.

12. The implantable cardioverter defibrillator device according to claim 1, wherein the generator device comprises a further, second antenna operatively connected to the communication circuitry for transmitting communication signals to and/or receiving communication signals from the external device in said frequency range above 2 GHz.

13. The implantable cardioverter defibrillator device according to claim 1, wherein a sensing arrangement for sensing electrocardiogram signals, wherein at least one electrode pole of said sensing arrangement is arranged on said at least one lead.

14. The implantable cardioverter defibrillator device (according to claim 13, wherein said sensing arrangement includes at least three electrode poles, a processing circuitry of the generator device being configured to sense electrocardiogram signals using different pairs of electrode poles of said at least three electrode poles.

15. A method for operating an implantable cardioverter defibrillator device, said implantable cardioverter defibrillator device comprising a generator device having a shock generation circuitry for producing an electrical shock pulse for performing a defibrillation therapy, the method comprising:
establishing, using a communication circuitry of the generator device, a communication connection to an external device in a frequency range above 2 GHz, wherein communication signals are transmitted to and/or received from the external device in said frequency range above 2 GHz using at least one antenna operatively connected to the communication circuitry and arranged on at least one lead connected to the generator device, the at least one lead comprising a shock electrode for emitting said electrical shock pulse.
